(19) United States
(12) Patent Application Publication
(10) Pub. No.: US 2019/0374743 A1
(43) Pub. Date: Dec. 12, 2019

(54) Infant Soothing Device

(71) Applicant: Kay Spence, Conley, GA (US)

(72) Inventor: Kay Spence, Conley, GA (US)

(21) Appl. No.: 16/545,210

(22) Filed: Aug. 20, 2019

Related U.S. Application Data
(63) Continuation-in-part of application No. 14/933,702, filed on Nov. 5, 2015, now abandoned.
(60) Provisional application No. 62/079,756, filed on Nov. 14, 2014.

(51) Int. Cl. A61M 21/02; A61H 7/00; A61H 23/02; A61H 23/00

(52) U.S. Cl. CPC A61M 21/02; A61H 7/004; A61M 2021/0022; A61H 23/006; A61H 23/0254

(57) ABSTRACT

An infant soothing device to provide a comforting and soothing touch to ease an infant to sleep. The infant soothing device includes an arm portion having a hand portion that can be placed against an infant. The arm portion is attached to an attachment arm via a maneuverable joint. The attachment arm is removably attached to the supporting structure, such as a crib or a stroller via a fastener thereon. The arm portion includes an actuating assembly that can actuate the arm portion in several modes of operation that can promote sleep for an infant. The actuating assembly includes a control panel that is electrically connected to a pair of motors that control the maneuverable joint. The control panel includes a plurality of switches that allow a user to activate and deactivate the actuating assembly and the modes of operation.

### CROSS REFERENCE TO RELATED APPLICATIONS

This application is a Continuation-In-Part of U.S. patent application Ser. No. 14/933,702 filed on Nov. 5, 2015, which claims the benefit of U.S. Provisional Application No. 62/079,756 filed on Nov. 14, 2014. The above identified patent applications are herein incorporated by reference in their entirety to provide continuity of disclosure.

### BACKGROUND OF THE INVENTION

The present invention relates to infant accessories. More particularly, the present invention provides an infant soothing device that includes an arm portion that is configured to provide a comforting and soothing touch to an infant to ease the infant to sleep. The arm portion is actuated by a pair of motors and includes several modes of operation that can be initiated by a user.

Typically, when children are sleeping or napping, these are precious moments for a parent or caregiver to relax and decompress. However, putting an infant to sleep is not always easy, and can be challenging. These struggles with the infant are often characterized by fits and fights, which often can be taxing an take a toll on any parent or caregiver. One of the most helpful ways of putting kids to bed is to establish a routine. Generally, a routine involves establishing a bedtime and improving the sleep environment to eliminate any distractions that may hinder him or her from falling asleep.

One of the hardships of establishing a routine, however, is that many infants require a soothing and comforting touch from their parent or caregiver for an extended period of time in order to fall asleep. These parents or caregivers are often forced to stand by a bed or a crib for extended periods of time massaging their infant's torso or back to ease him to sleep. This process can be taxing, and therefore, there exists a need for a device that mimics the soothing and comforting touch of a parent or caregiver to alleviate the stress of placing an infant to bed.

Devices have been disclosed in the prior art that relate to baby soothing devices. These include devices that have been patented and published in patent application publications. These devices, however, fail to provide an infant soothing device comprising an arm portion having a hand portion that can operate in various modes to soothe an infant, wherein the arm portion can be removably secured to a support via a fastener.

In light of the devices disclosed in the known art, it is submitted that the present invention substantially diverges in design elements from the known art and consequently it is clear that there is a need in the art for an improvement to existing infant soothing devices. In this regard, the instant invention substantially fulfills these needs.

### SUMMARY OF THE INVENTION

In view of the foregoing disadvantages inherent in the known types of infant soothing devices now present in the known art, the present invention provides an infant soothing device wherein the same can be utilized for mimicking a comforting touch of a caregiver to allow a caregiver to sleep, while also promoting sleep for the infant.

It is therefore an object of the invention to provide a new and improved infant soothing device that has all of the advantages of the prior art and none of the disadvantages.

Another object of the present invention is to provide a new and improved infant soothing device including an attachment portion attached to an arm portion via a maneuverable joint.

Yet another object of the present invention is to provide a new and improved infant soothing device, wherein said attachment portion includes a fastener configured to removably attach to a support structure.

Still yet another object of the present invention is to provide a new and improved infant soothing device, wherein said attachment portion includes a fastener configured to removably attach to a support structure.

A further object of the present invention is to provide a new and improved infant soothing device, wherein the arm portion supports an actuating assembly having a motor configured to actuate said arm portion and said hand portion in various modes of operation.

Yet a further object of the present invention is to provide a new and improved infant soothing device, wherein said several modes of operation including patting, massaging, and stroking.

An additional object of the present invention is to provide a new and improved infant soothing device, wherein said actuating assembly includes a control panel configured to activate said actuating assembly and the several modes of operation.

Still yet another object of the present invention is to provide a new and improved infant soothing device, wherein the device may be readily fabricated from materials that permit relative economy and are commensurate with durability.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a side perspective view of the infant soothing device.

FIG. 2 shows a side perspective view of the infant soothing device as removably attached to a support structure.

FIG. 3 shows a schematic diagram of the actuating assembly of the infant soothing device.

### DETAILED DESCRIPTION OF THE INVENTION

Reference is made herein to the attached drawings. Like reference numerals are used throughout the drawings to depict like or similar elements of the infant soothing device. For the purposes of presenting a brief and clear description of the present invention, the preferred embodiment will be discussed as used for providing a comforting touch for infants to promote sleep. The figures are intended for representative purposes only and should not be considered to be limiting in any respect.

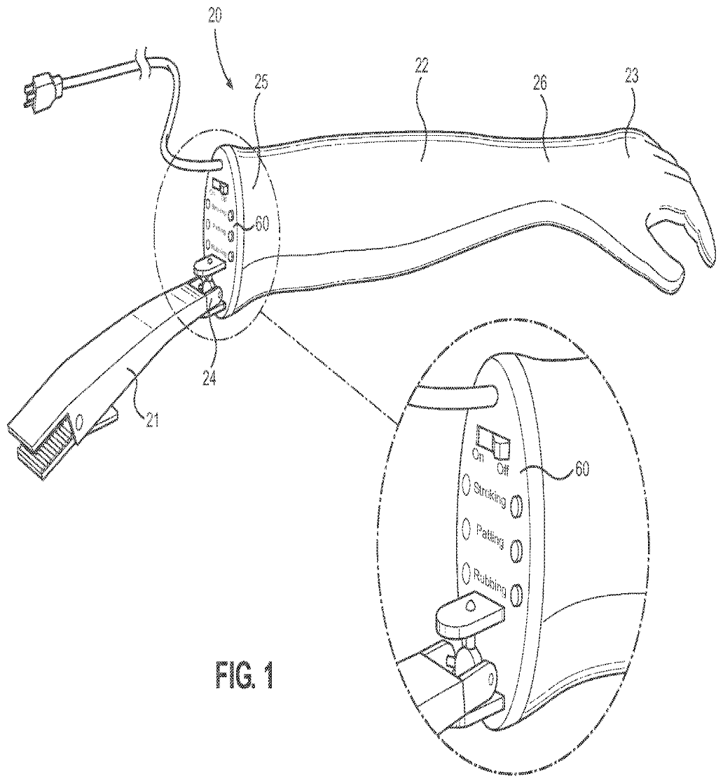

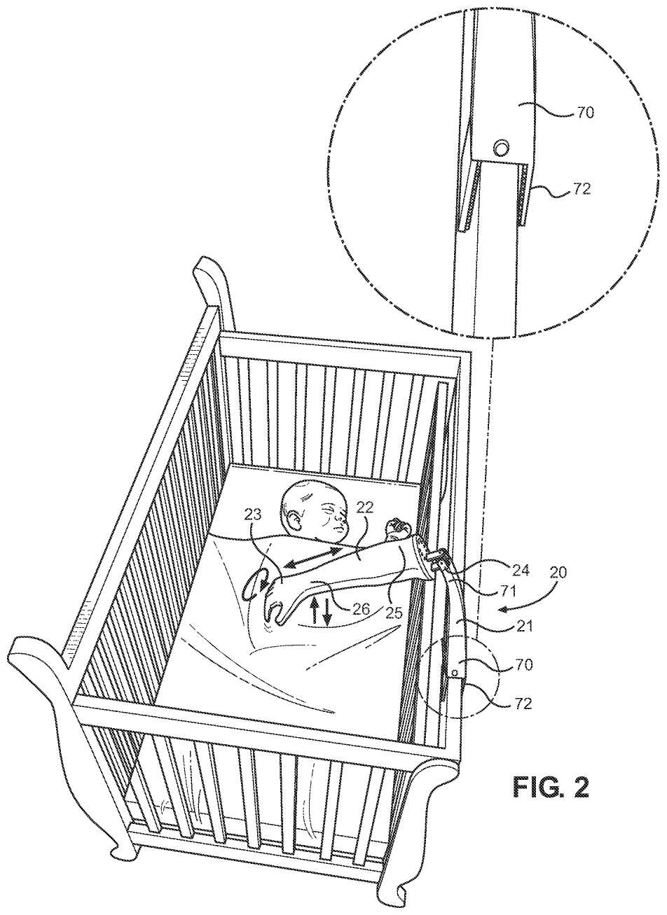

Referring now to FIGS. 1 and 2, there are shown a pair of side perspective views of the infant soothing device. The infant soothing device 20 is configured to mimic the comforting touch of a caregiver in order to sooth infants and other young children. In this way, the infant soothing device 20 allows these infants to fall asleep faster and more peacefully. The infant soothing device 20 comprises an attachment portion 21 that is attached to an arm portion 22. The arm portion 22 includes a hand portion 23 that is configured to be placed against an infant. It is contemplated that the hand portion 23 is preferably placed against an infant's back or stomach. The hand portion 23 is substantially hand-shaped including a palm portion and five finger portions that extend therefrom. Preferably, the five finger portions are partially curved to provide a gentle touch. In some embodiments, the arm portion 22 and the hand portion 23 further comprises a silicon covering thereon, wherein the silicon covering providing a cushioning effect that mimics the sensation of the touch of a person.

The arm portion 22 includes an actuating assembly having an internal motor configured to provide several modes of operation. These modes of operation allow for the arm portion 22, and therefore the attached hand portion 23, to be moved in motions such as patting, massaging, and stroking. These modes of operation allow for the infant to fall asleep without the need of a caregiver, thereby allowing the caregiver to receive proper rest. The arm portion 22 is movably attached to the attachment portion 21 via a maneuverable joint 24, which allows for the arm portion 22 and the hand portion 23 to provide various modes of operation.

In the illustrated embodiment, the maneuverable joint 24 further comprises a first bracket affixed to a distal end of the attachment portion 21, wherein a first axle extends across the first bracket. In this embodiment, the first axle is affixed to a ball joint, such that rotation of the first axle about a longitudinal axis thereof causes the ball joint to rotate about the longitudinal axis of the first axle. In this way, the force generated thereby is transferred through the first bracket, causing the arm portion 22 to rotate about the longitudinal axis of the first axle.

Similarly, in the illustrated embodiment, the maneuverable joint 24 further comprises a second bracket affixed to a proximal end of the arm portion 22, wherein a second axle extends across the second bracket. In this embodiment, the second axle is affixed to a ball joint, such that rotation of the second axle about a longitudinal axis thereof causes the ball joint to rotate about the longitudinal axis of the second axle. In this way, the force generated thereby is transferred through the second bracket, causing the arm portion 22 to rotate about the longitudinal axis of the second axle. In the illustrated embodiment, the first and second axles are disposed perpendicularly to each other, allowing independent rotation of the arm portion 22 about perpendicular axes.

Alternatively, other bracket assemblies are contemplated, wherein the maneuverable joint 24 comprises a ball and socket joint, wherein the first bracket is affixed to a socket, within which a ball affixed to the second bracket secures. In such an embodiment, a plurality of friction motors within the arm portion 22 are operably connected to rotational elements disposed on an interior surface of the socket configured to rotate the ball secured within the socket. In this alternate embodiment, the rotational elements are disposed perpendicularly within the socket, allowing the ball to be rotated along perpendicular axes, similarly to the first and second axle embodiment herein described elsewhere.

In some embodiments, the internal motor comprises a pair of motors, each independently coupled to one of the first and second axles. The pair of motors can be coupled to the first and second axles using a variety of methods, including, but not limited to, belts, gears, or other similar mechanical linkages known in the art to operably connect axles or other moving parts with a motor. In this way, each motor of the pair of motors is configured to reversibly rotate the arm portion 22 about independent perpendicular axes, allowing for the arm portion 22 to be rotated in either direction about the first axle when a first motor of the pair of motors is activated, and in either direction about the second axle when a second motor of the pair of motors is activated. When both motors of the pair of motors are activated, the arm portion 22 can simultaneously rotate about the first and second axles. In these embodiments, an internal processor or controller is operably connected to the pair of motors and can be configured to alternate the directions of rotation for each of the pair of motors, such that the arm portion 22 can be maneuvered through alternating directions of movement in a set pattern.

The arm portion 22 includes a first end 25 and a second end 26, wherein the first end 25 is attached to the attachment portion 21 via the maneuverable joint 24. In some embodiments, the arm portion 22 comprises a telescopic configuration so that the arm portion 22 can be extended to a desired length. In alternate embodiments, the arm portion 22 comprises a fixed length. The second end 26 includes the hand portion 23. The maneuverable joint 24 is configured to allow for the arm portion 22 to be moved in a variety of motions including, but not limited to, vertical motions, horizontal motions, and circular motions. Preferably, the maneuverable joint 24 is a ball and socket joint, however, other maneuverable joints, such as a universal joint that are suitable for achieving the advantages described in this present disclosure are likewise contemplated.

The internal motor is configured to actuate the arm portion 22 in various movements to move the arm portion 22 in different modes of operation. Specifically, when the patting mode of operation is selected, the first motor of the pair of motors is activated, causing the arm portion 22 to rotate about the longitudinal axis of the first axle as the force generated by the first motor is transferred through the first axle, into h first bracket, and through the maneuverable joint 24. In this way, the arm portion 22 move upwardly and downwardly relative to the attachment portion 21, resulting in a patting motion. Similarly when the stroking mode of operation is selected, the second motor of the pair of motors activates, causing the force arm portion 22 to rotate about the longitudinal axis of the second axle as the force generated is transferred through the maneuverable joint 24 to cause the arm portion 22 to move perpendicular to the motion of the patting mode of operation. Finally, when the massaging mode of operation is selected, both the first and second motors activate, resulting in a circular motion of the arm portion 22 as the forces generated by the pair of motors cause simultaneous rotation about the first and second axles. Furthermore, in some embodiments, the massaging mode of operation further includes a vibration action. In this embodiment, the arm portion further comprises a vibration motor therein, wherein the vibration motor is configured to activate when the massaging mode of operation is selected, thereby causing the arm portion 22 to vibrate. In some embodiments, the frequency of vibration of the vibration motor can be controlled via an intensity control disposed on a control panel 60 disposed on an outer face of the arm portion 22. In this way, the user can selectively adjust the vibration frequency to a desired setting. The internal motor is electrically connected to the control panel 60 to provide the ability to select and change the mode of operation as desired.

The attachment portion 21 includes a proximal end 70 and a distal end 71, wherein the proximal end 70 includes a fastener 72 and the distal end 71 is maneuverably attached to the first end 25 of the arm portion 22 via the maneuverable joint 24. The fastener 72 is configured to allow for the infant soothing device 20 to be removably attached to a support structure such as a stroller, a crib, or other similar items. Preferably, the fastener 72 is a mounting bracket that is configured to removably attach to the aforementioned items. The mounting bracket is substantially U-shaped to allow for removable attachment. In the illustrated embodiment, the fastener 72 comprises a spring biased clamp, wherein the clamp is biased towards a closed position, such that the fastener 72 can be removably secured to a crib or the like. Further, in the shown embodiment, the fastener 72 comprises a plurality of teeth disposed on an interior surface of each of the clamp portions, such that a greater surface area for frictional engagement with a crib railing can be achieved. It is contemplated that those of ordinary skill in the art will immediately recognize a variety of other suitable fasteners that achieve the results and advantages described herein; these variations and modifications are deemed within the scope and spirit of the inventive embodiments of the present invention.

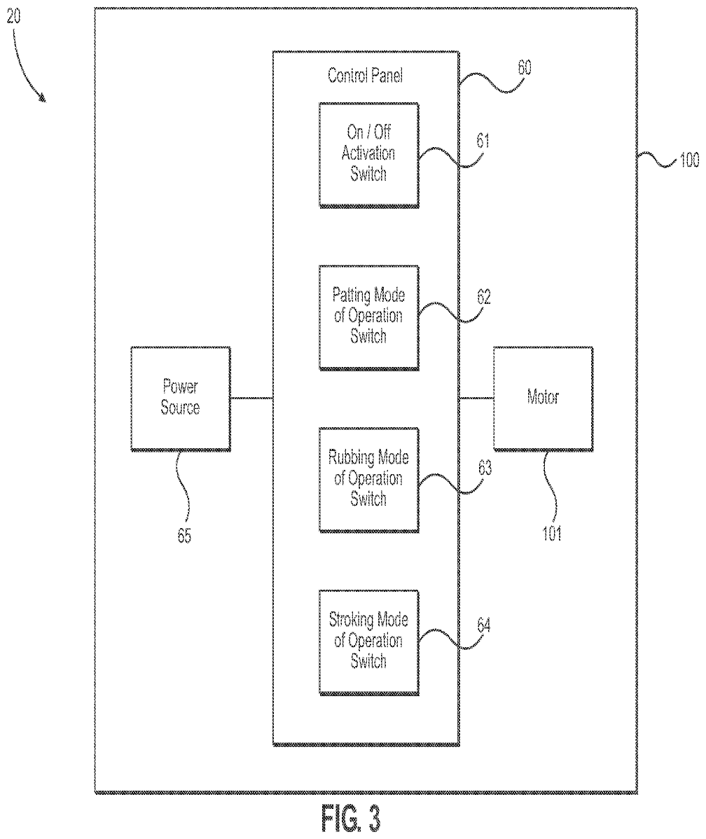

Referring now to FIG. 3, there is shown a schematic diagram of the infant soothing device. The infant soothing device 20 includes an actuating assembly 100 that allows for the motor 101 to actuate the arm portion and hand portion in several modes of operation. The actuating assembly 100 includes the control panel 60 that is configured to allow a user to choose the mode of operation for the infant soothing device 20. The control panel 60 includes an on/off activation switch 61, a patting mode of operation switch 62, a massaging mode of operation switch 63, and a stroking mode of operation switch 64. The on/off activation switch 61 is electrically connected to a power source 65 to provide voltage to operate the infant soothing device 20. Preferably, the power source 65 comprises one or more disposable or rechargeable batteries.

The switches 62, 63, 64 are electrically connected to the motor 101 via a controller, such that actuation of the switches 62, 63, 64 sends a signal to the controller to activate the motor 101, or pair of motors as previous described herein, in a variety of preset patterns. These preset patterns are referred to throughout this disclosure as the patting mode of operation, the massaging mode of operation, and the stroking mode of operation. The switch 62 is configured to activate and deactivate the patting mode of operation. The switch 63 is configured to activate and deactivate the massaging mode of operation. The switch 64 is configured to activate and deactivate the stroking mode of operation. It is contemplated that those of ordinary skill in the art will readily envision other suitable modes of operation that are appropriate for soothing an infant. It is further contemplated that these additions, modifications, and variations include their own respective switches on the control panel that allow for the activation and deactivation of the envisioned mode of operation.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An infant soothing device, comprising:
an arm portion having a first end and a second end, wherein the first end includes a hand portion configured to be placed against an infant's back or torso;
an attachment portion movably connected to the second end of the arm portion via a maneuverable joint, wherein the attachment portion is configured to be removably fastened to a support structure;
wherein the maneuverable joint comprises a first bracket extending from a distal end of the attachment portion having a first axle extending therebetween and a second bracket extending from a proximal end of the arm portion having a second axle extending therebetween;
an actuating assembly enclosed within said arm portion, the actuating assembly comprising a first motor and a second motor;
wherein the first motor is operably connected to the first axle, such that when the first motor is actuated, rotational forces generated by the first motor are transferred to the first axle, causing the first axle to rotate about a longitudinal axis thereof;
wherein the second motor is operably connected to the second axle, such that when the second motor is actuated, rotational forces generated by the second motor are transferred to the second axle, causing the second axle to rotate about a longitudinal axis thereof;
wherein the first axle and the second axle intersect perpendicularly at a ball joint, such that rotational forces distributed through the first and second axles are transferred through the maneuverable joint and into the arm portion, thereby maneuvering the arm portion.

2. The infant soothing device of claim 1, wherein the maneuverable joint is configured to simultaneously rotate about each of the first and second axles, thereby moving the arm portion and the hand portion in a circular pattern.

3. The infant soothing device of claim 1, wherein:
said actuating assembly further comprises a control panel having a plurality of switches electrically connected to said motor;
wherein said plurality of switches is configured to provide voltage to said actuating assembly.

4. The infant soothing device of claim 3, wherein said plurality of switches include:
an on/off activation switch;
a patting mode of operation switch configured to activate the first motor when actuated, causing the arm portion to alternatingly rotate about the first axle;
a stroking mode of operation switch configured to activate the second motor when actuated, causing the arm portion to alternatingly rotate about the second axle; and
a massaging mode of operation switch configured to simultaneously activate the first and second motors, causing the arm portion to rotate about the first and second axles resulting in a circular motion.

5. The infant soothing device of claim 1, wherein said actuating assembly further includes a power source.

6. The infant soothing device of claim 1, wherein the attachment portion comprises a mounting bracket for securement to the support structure.

7. The infant soothing device of claim 1, further comprising a silicon covering disposed over the arm portion.

8. The infant soothing device of claim 1, wherein the hand portion further comprises plurality of arcuate finger portions extending therefrom.

9. The infant soothing device of claim 1, wherein a length of the arm portion is telescopically adjustable.

10. The infant soothing device of claim 4, wherein the arm portion further comprises a vibration motor therein, wherein the vibration motor is configured to vibrate when the massaging mode of operation switch is actuated.

11. The infant soothing device of claim 10, further comprising an intensity control disposed on the control panel, wherein the intensity control is configured to adjust the frequency of vibration generated by the vibration motor.

12. The infant soothing device of claim 6, wherein the mounting bracket comprises a clamp configured to removably receive the support structure therein, wherein the clamp is spring-biased towards a closed position.

13. The infant soothing device of claim 12, wherein a plurality of teeth extend from an interior surface of the clamp.
